(19) United States
(12) Patent Application Publication
(10) Pub. No.: US 2025/0369915 A1
(43) Pub. Date: Dec. 4, 2025

(54) NON-DESTRUCTIVE BIOPROFILING DEVICE INCLUDING SPATIAL RESOLUTION, AND METHOD OF OPERATING SAME

(71) Applicant: PROVALABS, INC., Seoul (KR)

(72) Inventor: Jaegon KIM, Seoul (KR)

(21) Appl. No.: 19/107,036

(22) PCT Filed: Aug. 25, 2023
(86) PCT No.: PCT/KR2023/012658

§ 371 Date: Feb. 27, 2025

(30) Foreign Application Priority Data
Aug. 30, 2022 (KR) 10-2022-0109218
Aug. 23, 2023 (KR) 10-2023-0110802

(51) Int. Cl. G01N 27/327; G01N 27/02; G01N 33/50

(52) U.S. Cl. CPC G01N 27/327; G01N 27/02; G01N 33/5005

(57) ABSTRACT

An impedance measurement device according to an embodiment of the present disclosure includes three or more electrodes electrically connected to biological tissue; a power supply unit including a first terminal and a second terminal, and supplying power through the first terminal and the second terminal; a multiplexing circuit selecting at least some of the electrodes and connecting to the first terminal and the second terminal; and a controller providing an electrode selection signal to the multiplexing circuit, the electrode selection signal including information on the electrodes to be connected to the first terminal and the second terminal, wherein an impedance of the biological tissue is measured by measuring an electrical signal between the first terminal and the second terminal.

### TECHNICAL FIELD

Embodiments of the present invention relate to a non-destructive bio-profiling device including spatial resolution, and more particularly, to an impedance measurement device for measuring the impedance of biological tissue and a method of operating the same.

### BACKGROUND ART

Recently, as time and cost inefficiencies in new drug development have rapidly increased, the need for biological models that can more precisely predict drug efficacy and toxicity is increasing. Currently, two-dimensional (2D) cell line models are mainly used in early drug screening. However, 2D cell culture has various limitations in accurately replicating in vivo phenomena, and most new drug candidates that exhibited low toxicity and high efficacy in preclinical trials fail to progress to clinical trials.

Micro-physiological systems (MPS), such as organoids and organ-on-a-chips, embed a 3D cell culture model through culturing appropriate cell lines or primary cells on a 3D structure to simulate the body organ, function, or phenomenon to be tested. Recently, advancements in Multi Organ-on-Chip, which involves multiple identical organ-on-chip systems operating in parallel, and Human-on-Chip or Body-on-Chip, which integrates different organs-on-a-chip, have been accelerating innovation in drug development. These technologies aim to create a more human-like physiological environment, enabling the faster development of safer, more effective, and cost-efficient drugs with fewer side effects.

As with the characteristics of living organisms that are difficult to secure uniformity, in order to implement effective drug screening using micro-physiological systems, the uniformity of individual micro-physiological systems must be confirmed by non-destructive testing methods, and electrical measurement methods are an effective way to realize this. Among them, Transepithelial Electrical Resistance (TEER) measurement is an important tool for evaluating the barrier function of the cell layer in cell culture models and belongs to a representative non-destructive testing method. The TEER measurement is a method of measuring the resistance or low-frequency impedance of biological tissue, and is widely used as a non-destructive analysis method that quantitatively measures the tight junction of biological tissue. The stronger the binding between the cells that form the tissue, the higher the resistance value is measured by blocking the movement of the electrolyte, and if the binding between the tissues weakens due to physicochemical stimulation or aging, the resistance value decreases, so this can be used in drug screening to evaluate the toxicity and efficacy.

However, the existing TEER measurement can only evaluate an object as a whole, making it difficult to accurately determine local differences on its surface or identify the location of damage to the cell layer. In addition, existing devices have problems with error-causing factors in the measurement environment, inconvenience due to complex protocols required for measurement preparation, and poor repeatability and reproducibility.

Therefore, there is a need for a new TEER measurement device that can solve existing problems.

### DISCLOSURE

### Technical Problem

An embodiment of the present invention provides an impedance measurement device capable of analyzing electrical characteristics according to a position in biological tissue, and an operating method thereof.

### Technical Solution

An impedance measurement device according to an embodiment of the present invention may include three or more electrodes electrically connected to biological tissue; a power supply unit including a first terminal and a second terminal, and supplying power through the first terminal and the second terminal; a multiplexing circuit selecting at least some of the electrodes and connecting to the first terminal and the second terminal;

and a controller providing an electrode selection signal to the multiplexing circuit, the electrode selection signal including information on the electrodes to be connected to the first terminal and the second terminal, wherein an impedance of the biological tissue is measured by measuring an electrical signal between the first terminal and the second terminal.

An operating method of a impedance measurement device according to an embodiment of the present invention may include: electrically connecting three or more electrodes to biological tissue (step 1); selecting at least some of the electrodes through a multiplexing circuit and connecting to a first terminal and a second terminal (step 2); measuring an impedance of the biological tissue through the first terminal and the second terminal (step 3); changing an electrode connected to at least one of the first terminal and the second terminal (step 4); and measuring the impedance of biological tissue through the first terminal and the second terminal (step 5).

### Advantageous Effects

According to the present invention, an impedance measurement device capable of analyzing electrical characteristics according to a position in biological tissue, and an operating method thereof are provided.

### DESCRIPTION OF DRAWINGS

FIG. 1 is a block diagram illustrating an impedance measurement device according to an embodiment of the present invention.

FIG. 2 is a diagram illustrating a multiplexing circuit of FIG. 1 in more detail.

FIG. 3 is a diagram illustrating a sample holder and an upper cover of the impedance measurement device according to an embodiment of the present invention.

FIG. 4 is a flowchart illustrating a method of operating the impedance measurement device according to an embodiment of the present invention.

FIG. 5 illustrates an image generated by the impedance measurement device according to an embodiment of the present invention.

FIG. 6 is a diagram comparing biological tissue to be measured with an image generated by the impedance measurement device according to an embodiment of the present invention.

### BEST MODES OF THE INVENTION

The structural or functional descriptions of the embodiments disclosed in this specification or the application are merely exemplified for the purpose of describing the embodiments according to the technical idea of the present invention, and the embodiments according to technical idea of the invention may be implemented in various forms in addition to the embodiments disclosed in the specification or the application, and the technical idea of this invention is not to be construed as being limited to the embodiments described in this specification or in the application.

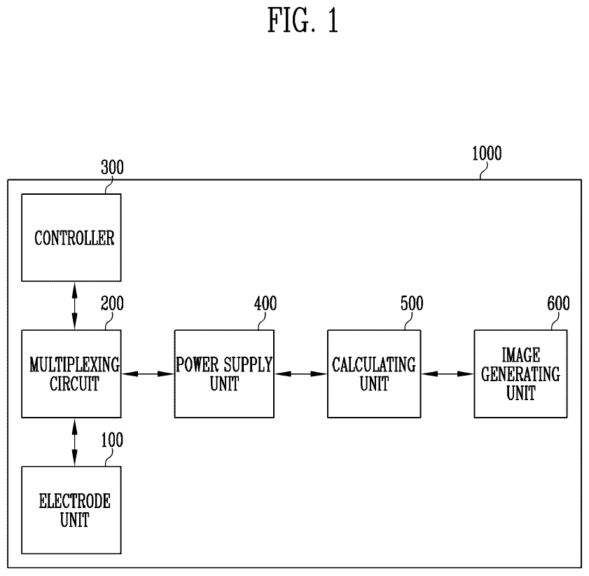

FIG. 1 is a block diagram illustrating an impedance measurement device according to an embodiment of the present invention.

Referring to FIG. 1, an impedance measurement device 1000 includes an electrode unit 100, a multiplexing circuit 200, a controller 300, and a power supply unit 400.

The electrode unit 100 may include three or more electrodes. The electrodes may be electrically connected to biological tissue to be measured. In the present specification, being electrically connected may refer to direct contact or connection with each other through a medium having electrical conductivity. In an embodiment, electrodes may be in direct contact with the biological tissue to be measured. In another embodiment, the electrodes may be electrically connected to the biological tissue to be measured through an electrolyte. In addition, in this specification, it can be understood that the electrical connection between the electrodes and the biological tissue is sufficient if current may flow as power is applied later, and there is no need for power to be applied at the moment of connection.

In an embodiment, the biological tissue may be tissue detached from an organism or a cultured cell culture. For example, the cell culture may be a spheroid or an organoid. In an embodiment, the electrodes may be replaceable electrodes.

The multiplexing circuit 200 may select at least a portion of the electrodes and connect the electrodes to the power supply unit 400, and more specifically, may connect the electrodes to terminals of the power supply unit 400. The multiplexing circuit 200 may receive an electrode selection signal from the controller 300, and may select electrodes to be connected to the respective terminals based on the received electrode selection signal.

The controller 300 may control the multiplexing circuit 200. In an embodiment, the controller 300 may provide the electrode selection signal to the multiplexing circuit 200, and the electrode selection signal may include information on the electrodes to be connected to respective terminals of the power supply unit 400. In an embodiment, the controller 300 may control the multiplexing circuit 200 to change the electrodes connected to the respective terminals. For example, the controller 300 may provide a new electrode selection signal to the multiplexing circuit 200 that includes information about electrodes to be newly connected to the terminals, and the multiplexing circuits 200 may select new electrodes accordingly and connect the electrodes to the respective terminals.

In an embodiment, the controller 300 may change the electrodes connected to the respective terminals according to a predetermined order. That is, the impedance measurement device 1000 may include a memory (not shown), and may include information on the order of electrode combinations connected to the terminals in the memory. The controller 300 may provide the electrode selection signal to the multiplexing circuit 200 based on information about the order of electrode combinations stored in the memory (not shown).

The power supply unit 400 may include a first terminal and a second terminal. In an embodiment, one or more electrodes may be electrically connected to each of the first terminal and the second terminal. In an embodiment, the electrodes respectively connected to the first terminal and the second terminal may be different electrodes from each other. The power supply unit 400 may supply power through the first terminal and the second terminal, and the power supplied from the power supply unit 400 may be supplied to the biological tissue to be measured through the electrodes connected to the first terminal and the second terminal. The power source may be, for example, a current or a voltage, and each of the current or the voltage may be a direct current or an alternating current. In an embodiment, the first terminal may include a first current terminal and a first voltage terminal, and the second terminal may include a second current terminal and a second voltage terminal. In an embodiment, the same electrode may be connected to the first current terminal and the first voltage terminal, but is not limited thereto, and in another embodiment, different electrodes may be connected to the first current terminal and the first voltage terminal. In an embodiment, the same electrode may be connected to the second current terminal and the second voltage terminal, but is not limited thereto, and in another embodiment, different electrodes may be connected to the second current terminal and the second voltage terminal. In addition, in an embodiment, electrodes that are not connected to the first terminal and the second terminal may be electrically insulated.

The impedance measurement device 1000 may measure impedance of the biological tissue by measuring an electrical signal between the first terminal and the second terminal. In an embodiment, the power supply unit 400 may apply a current through the first current terminal and the second current terminal, and the impedance of the biological tissue may be measured by measuring a voltage between the first voltage terminal and the second voltage terminal according to the applied current. In an embodiment, the applied current may be 10 mA or less, and more specifically, may be 0.5 μA to 20 μA, but is not limited thereto, and a current of a different magnitude may be applied according to the size and state of the object to be measured. In another embodiment, the power supply unit 400 may apply a voltage through the first voltage terminal and the second voltage terminal, and the impedance of the biological tissue may be measured by measuring a current flowing through the first current terminal and the second current terminal according to the applied voltage. In an embodiment, the applied voltage may be 30 V or less, and more specifically, may be 50 mV to 10 V, but is not limited thereto, and a voltage of a different magnitude may be applied according to the size and state of the measurement object.

In an embodiment, the impedance measurement apparatus 1000 may further include a calculating unit 500. The calculating unit 500 may calculate electrical characteristics according to the position in the biological tissue based on the measured impedance and the positions of the electrodes. In an embodiment, the electrical characteristics according to the position may be expressed as an impedance value, an electrical conductivity value, or the like, but are not limited to a specific example.

For example, in one measurement sequence in which one combination of the electrodes is connected to the first terminal and the second terminal, the positions of the electrodes connected to the first and second terminals and the impedance values measured in the measurement sequence may be stored in the impedance measurement device 1000. As a plurality of measurement sequences are repeated while changing the combination of the electrodes, a plurality of impedance values and positions of the electrodes corresponding thereto may be stored in the impedance measurement device 1000, and the calculating unit 500 may calculate the electrical characteristics according to the position in the biological tissue based on the plurality of impedance values stored in the impedance measurement device 1000 and the positions of the electrodes corresponding thereto. In an embodiment, the calculating unit 500 may further calculate the electrical characteristics by using a correction coefficient for considering the asymmetric and non-uniform shape of the biological tissue.

In an embodiment, the impedance measurement device 1000 may further include an image generating unit 600. The image generating unit 600 may generate an image indicating the electrical characteristics of the biological tissue based on the electrical characteristics according to the position calculated by the calculating unit 500.

Accordingly, the impedance measurement device 1000 according to an embodiment of the present invention may provide multidimensional electrical characteristic measurement data for the biological tissue. That is, the impedance measurement device 1000 according to an embodiment of the present invention provides spatial resolution, so that it may be possible to measure non-uniform or local changes in the biological tissue.

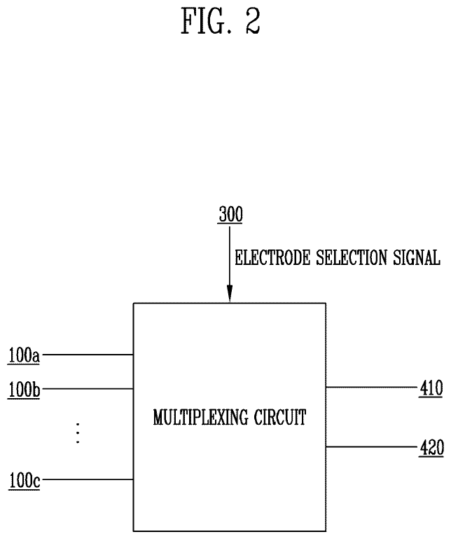

FIG. 2 is a diagram illustrating the multiplexing circuit of FIG. 1 in more detail.

Referring to FIG. 2, electrodes 100a, 100b, and 100c may be connected to the multiplexing circuit 200. Although three electrodes 100a, 100b, and 100c are shown in FIG. 2, the number of electrodes connected to the multiplexing circuit 200 is not limited thereto, and may be four or more.

The multiplexing circuit 200 may receive the electrode selection signal from the controller 300, may select electrodes to be connected to a first terminal 410 and a second terminal 420 based on the electrode selection signal, and may connect the selected electrodes to the first terminal 420 and the second terminal 410.

As controller 300 provides the new electrode selection signal to the multiplexing circuit 200, the multiplexing circuit 200 may change the electrodes connected to first terminal 410 and second terminal 420.

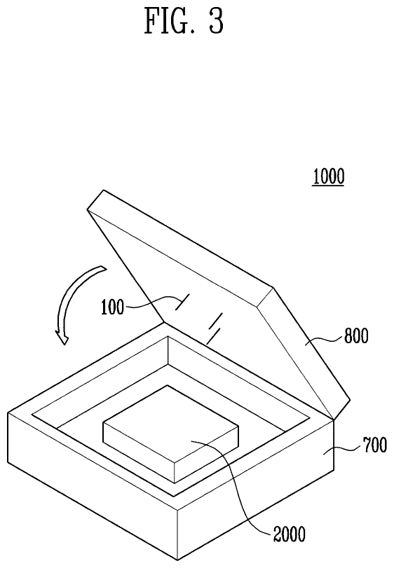

FIG. 3 is a diagram illustrating a sample holder and an upper cover of the impedance measurement device according to an embodiment of the present invention.

Referring to FIG. 3, the impedance measurement device 1000 may include a sample holder 700. The biological tissue may be disposed in the sample holder 700. In an embodiment, the biological tissue cultured in a transwell may be disposed in the sample holder.

In addition, the impedance measurement device 1000 may include an upper cover 800. The upper cover 800 may be disposed over the sample holder 700. In an embodiment, the sample holder 700 and the top cover 800 may be hinged to form a clam-shell structure, but are not limited to such a structure.

In an embodiment, as shown in FIG. 3, three or more electrodes of the impedance measurement device 1000 may be fixed to the upper cover 800. In another embodiment, some of the three or more electrodes of the impedance measurement device 1000 may be fixed to the upper cover 800, and others may be fixed to the sample holder 700. As the upper cover 800 is disposed over the sample holder 700, the electrodes may be electrically connected to biological tissue 2000. In an embodiment, as the electrodes are fixed to the upper cover 800 or the sample holder 700, the impedance measurement device 1000 may measure the impedance without any deviation according to the movement of the electrodes, thereby more accurately measuring the electrical characteristics of the biological tissue.

In addition, in an embodiment, the impedance measurement device 1000 may minimize noise generation by electrically shielding the internal space between the sample holder 700 on which the biological tissue is disposed and the upper cover 800, additionally including a vibration absorbing pad, or the like. In another embodiment, the impedance measurement device 1000 may further include a guarding circuit for removing noise.

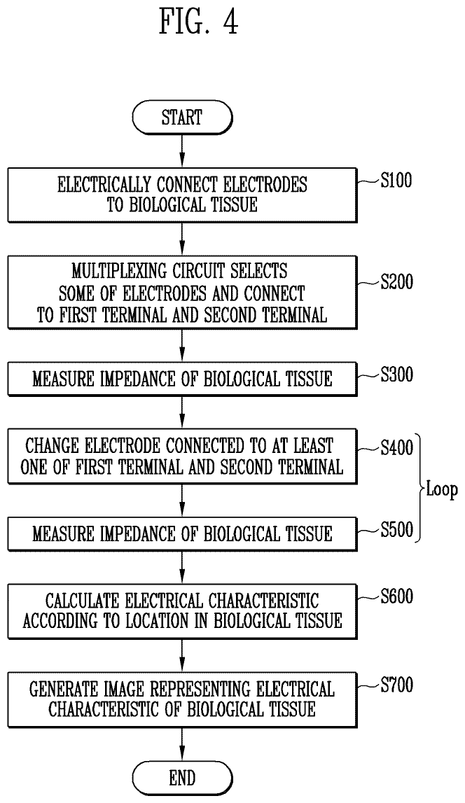

FIG. 4 is a flowchart illustrating a method of operating the impedance measurement device according to an embodiment of the present invention.

Referring to FIG. 4, the electrodes may be electrically connected to the biological tissue in operation S100. For example, as shown in FIG. 3, after the biological tissue 2000 is placed in the sample holder 700, the electrodes of the electrode unit 100 fixed to the upper cover 800 or the sample holder 700 may be electrically connected to the biological tissue 2000 by placing the upper cover 800 on the sample holder 700. That is, the electrodes fixed to the upper cover 800 or the sample holder 700 may directly contact the biological tissue 2000 or may be electrically connected to the biological tissue 2000 through an electrolyte.

In operation S200, the multiplexing circuit may select some of the electrodes and connect the electrodes to the first terminal and the second terminal. As shown in FIG. 2, the multiplexing circuit 200 may receive the electrode selection signal from the controller 300, and may select the electrodes to be connected to the first terminal and the second terminal based on the electrode selection signal.

In operation S300, the impedance of the biological tissue may be measured. The impedance of the biological tissue may be measured through the first terminal and the second terminal, and more particularly, may be measured by measuring the electrical signal between the first terminal and second terminal.

In operation S400, the electrode connected to at least one of the first terminal and the second terminal may be changed. For example, as shown in FIG. 2, the controller 300 may provide the new electrode selection signal to the multiplexing circuit 200, so that the multiplexing circuit 200 may change the electrodes connected to the first terminal and/or the second terminal. In an embodiment, at least some of the one or more electrodes respectively connected to the first terminal and the second terminal may be changed, but are not limited thereto, in another embodiment, only at least some of electrodes connected to the first terminal may be changed, and in yet another embodiment, only the at least some of electrode connected to the second terminal may be changed.

In operation S500, the impedance of the biological tissue may be measured again based on the changed electrodes connected to the first terminal and the second terminal. In an embodiment, operations S400 to S500 may be repeatedly performed. For example, information about the combination of electrodes changing according to the predetermined order may be stored in the impedance measurement device, and the controller 300 may repeatedly provide the new electrode selection signal to the multiplexing circuit 200 based on the information about the combination of electrodes changing according to the predetermined order.

In operation S600, the electrical characteristics according to the location in the biological tissue may be calculated. In an embodiment, the impedance measurement device may store position information of the electrodes connected to the first terminal and the second terminal and the impedance measurement values corresponding thereto for each of the sequences and calculate the electrical characteristics according to the positions in the biological tissue based on the position information of the electrodes and the impedance measurement values.

In operation S700, the image representing the electrical characteristics of the biological tissue may be generated. The image representing the electrical characteristics of the biological tissue may be generated based on the electrical characteristics according to the location in the biological tissue calculated in operation S600.

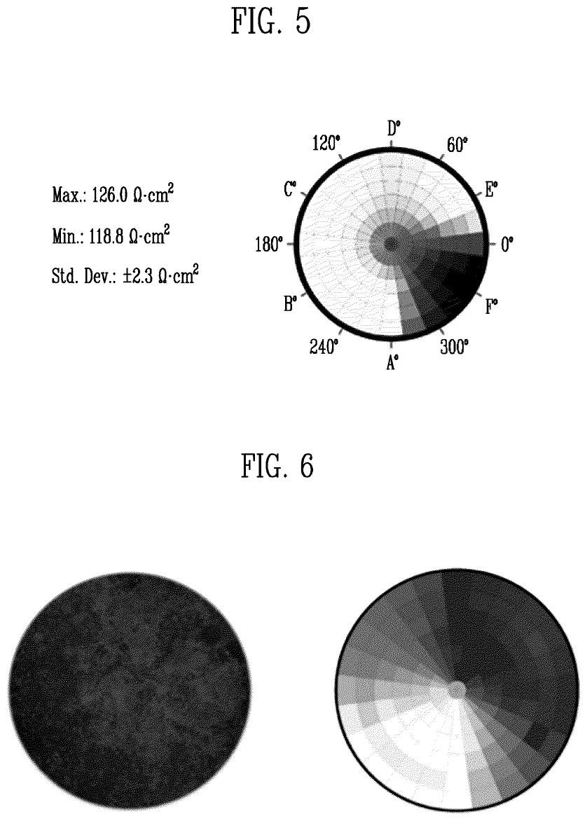

FIG. 5 illustrates the image generated by the impedance measurement device according to an embodiment of the present invention.

Referring to FIG. 5, the image generated through operation S700 of FIG. 4 may be confirmed. The impedance and electrical conductivity values were calculated for each location in the biological tissue, and displayed in different colors according to the electrical conductivity values. That is, the impedance measurement device according to an embodiment of the present invention may provide spatial resolution, and thus may be capable of measuring non-uniform or local changes in biological tissue.

In addition, as shown in FIG. 5, a minimum value and a maximum value of impedances for each position in the biological tissue may be calculated, and a standard deviation of impedance values for each position in the biological tissue may also be calculated. In an embodiment, information such as the minimum impedance value for each position and a corresponding position, the maximum impedance value for each location and a corresponding position and an average impedance value and the standard deviation in the biological tissue may be provided to a user through a display.

FIG. 6 is a diagram comparing the biological tissue to be measured with the image generated by the impedance measurement device according to an embodiment of the present invention.

Referring to FIG. 6, the left image is an image of a fluorescent staining (F-actin) result of an intestinal model in which an intestinal epithelial cell line (Caco-2) is cultured, and the right image is the image generated by the impedance measurement device according to an embodiment of the present invention.

Upon review the fluorescent staining result of the intestinal model, the places with low cell density are displayed in relatively dark colors. Upon review the image of the intestinal model by the impedance measurement device, a portion measured to have a low impedance value was displayed in a bright color, and a portion measured to have a high impedance value was displayed in a dark color. Since the lower the cell density, the lower the measured impedance value, as shown in FIG. 6, the image of the intestinal model by the impedance measurement device successfully simulates the cell density in the actual intestinal model.

That is, the impedance measurement device according to the embodiment of the present invention may measure low cell density and epithelial tissue damage at a specific location.

### DESCRIPTION OF SYMBOLS

100: ELECTRODE UNIT
200: MULTIPLEXING CIRCUIT
300: CONTROLLER
400: POWER SUPPLY UNIT
500: CALCULATING UNIT
600: IMAGE GENERATING UNIT
700: SAMPLE HOLDER
800: UPPER COVER
1000: IMPEDANCE MEASUREMENT DEVICE
2000: BIOLOGICAL TISSUE

1. An impedance measurement device comprising:
three or more electrodes electrically connected to biological tissue;
a power supply unit including a first terminal and a second terminal, and supplying power through the first terminal and the second terminal;
a multiplexing circuit selecting at least some of the electrodes and connecting to the first terminal and the second terminal; and
a controller providing an electrode selection signal to the multiplexing circuit, the electrode selection signal including information on the electrodes to be connected to the first terminal and the second terminal,
wherein an impedance of the biological tissue is measured by measuring an electrical signal between the first terminal and the second terminal.

2. The impedance measurement device according to claim 1, wherein the controller controls the multiplexing circuit to change the electrodes connected to the first terminal and the second terminal according to a predetermined order.

3. The impedance measurement device according to claim 1, wherein the first terminal includes a first voltage terminal and a first current terminal, and
wherein the second terminal includes a second voltage terminal and a second current terminal.

4. The impedance measurement device according to claim 3, wherein the power supply unit applies a current through the first current terminal and the second current terminal, and
wherein the impedance is measured by measuring a voltage between the first voltage terminal and the second voltage terminal.

5. The impedance measurement device according to claim 3, wherein the power supply unit applies a voltage to the first current terminal and the second current terminal, and
wherein the impedance is measured by measuring a current flowing through the first voltage terminal and the second voltage terminal.

6. The impedance measurement device according to claim 1, further comprising:
a calculating unit configured to calculate electrical characteristics according to a position in the biological tissue based on impedances measured from different electrodes and positions of the electrodes; and
an image generating unit configured to generate an image representing the electrical characteristics of the biological tissue based on the electrical characteristics according to the position.

7. The impedance measurement device according to claim 1, further comprising:
a sample holder in which the biological tissue is disposed; and
an upper cover on which at least some of the electrodes are fixed and disposed on the sample holder.

8. The impedance measurement device according to claim 7, wherein the sample holder and the upper cover are hinged together to form a clam-shell structure.

9. A method of operating an impedance measurement device, the method comprising:
electrically connecting three or more electrodes to biological tissue (step 1);
selecting at least some of the electrodes through a multiplexing circuit and connecting to a first terminal and a second terminal (step 2);
measuring an impedance of the biological tissue through the first terminal and the second terminal (step 3);
changing an electrode connected to at least one of the first terminal and the second terminal (step 4); and
measuring the impedance of biological tissue through the first terminal and the second terminal (step 5).

10. The method of claim 9, further comprising:
calculating electrical characteristics according to a position in the biological tissue based on the measured impedance and a position of the electrodes; and
generating an image representing the electrical characteristics of the biological tissue based on the electrical characteristics according to the position.
